(19) United States
(12) Patent Application Publication
(10) Pub. No.: US 2023/0364023 A1
(43) Pub. Date: Nov. 16, 2023

(54) DRUG COMPOSITIONS AND METHODS OF PREPARING THE SAME

(71) Applicant: Applied Materials, Inc., Santa Clara, CA (US)

(72) Inventors: Miaojun Wang, Santa Clara, CA (US); Jonathan Frankel, Los Gatos, CA (US); Pravin K. Narwankar, Sunnyvale, CA (US); Suneel Kumar Rastogi, Hillsborough, NJ (US); Shivkumar Chiruvolu, San Jose, CA (US); Fei Wang, Fremont, CA (US); Balaji Ganapathy, Maharashtra (IN); Shrikant Swaminathan, Santa Clara, CA (US)

(21) Appl. No.: 18/199,614

(22) Filed: May 19, 2023

Related U.S. Application Data
(63) Continuation of application No. PCT/US23/20721, filed on May 2, 2023.
(60) Provisional application No. 63/337,994, filed on May 3, 2022.
(60) Provisional application No. 63/337,995, filed on May 3, 2022.

(51) Int. Cl. A61K 9/50

(52) U.S. Cl. CPC A61K 9/501; A61K 9/5089

(57) ABSTRACT

Methods for providing an inorganic oxide coating to high aspect ratio particles containing an active pharmaceutical ingredient are described as are compositions containing such coated particles.

### CROSS-REFERENCE TO RELATED APPLICATIONS

This application is a by-pass continuation application of and claims the benefit of priority to PCT Application No. PCT/US2023/020721 filed on May 2, 2023, which claims priority to U.S. Provisional Application Nos. 63/337,994 and 63/337,995 filed on May 3, 2022, the contents of which are hereby incorporated by reference.

### TECHNICAL FIELD

This disclosure pertains to methods of preparing coated particles containing an active pharmaceutical ingredient, coated particles and pharmaceutical compositions containing such particles.

### BACKGROUND

It is of great interest to the pharmaceutical industry to develop improved formulations of active pharmaceutical ingredients (APIs). Formulation can influence the stability and bioavailability of the APIs as well as other characteristics of the APIs and the drug product (DP) containing the APIs.

In the manufacture of drug products it is desirable for particles containing an API to have an aspect ratio (maximum dimension/minimum dimension) near 1 (e.g., be nearly spherical). This is because such particles can be easier to handle. For example, they generally have better flowability and higher bulk density compared to otherwise similar particles having a higher aspect ratio (i.e., needle-shaped particles). In many cases, higher flowability and bulk density are associated with easier and safer handling of APIs containing particles during drug product manufacturing.

Poor flowability and low bulk density are challenges that are traditionally addressed by wet/dry granulation and blending with glidants. However, these processes often include multiple powder handling steps that increase manufacturing costs. Moreover, aqueous wet granulation may not be suitable for an API that is thermolabile or prone to moisture induced degradation. Dry granulation can be more acceptable in such circumstances but fails to sufficiently improve flowability and results in poor tableting properties such as compactability, compressibility and/or tabletability.

Direct compression is a desirable alternative for the manufacture of solid oral dosage forms because it includes fewer steps (blending, compression and, optionally, coating) and can be carried out as a continuous process. However, the ability to employ continuous, direct compression requires an API with good flowability, relatively high bulk density and good compactability.

Flowability and compactability can be improved by coating API particles, but spray coating and dip coating can yield particles with a thick coating that can reduce the ability to create dosage forms with a high drug load.

Many APIs naturally form needle-shaped particles with a high aspect ratio (maximum dimension/minimum dimension). There are at least three aspects of needle-shaped particles that make it difficult to create particles that flow reasonably freely and have an acceptable bulk density: adherence between particles based on surface energy, friction, and tangling. Importantly, many APIs that form needle-shaped particles actually form particles that are dendrites, i.e., they have branches that can increase tangling of particles. This makes it particularly difficult to achieve acceptable flowability and bulk density.

Thus, there is a considerable interest in controlling or altering the shape of API-containing particles to decrease aspect ratio and in formulating needle-shaped particles with a high aspect ratio to increase their flowability and/or bulk density.

### SUMMARY

Described herein is a method of preparing a pharmaceutical composition comprising inorganic oxide-coated particles comprising an organic active pharmaceutical ingredient (API) enclosed by a conformal layer of at least one inorganic oxide, the method comprising the sequential steps of: (a) providing particles comprising an organic active pharmaceutical ingredient wherein the particles have an aspect ratio (maximum dimension/minimum dimension) greater than 5; (b) performing atomic layer deposition to apply at least one inorganic oxide layer selected from metal oxides and metalloid oxide to the particles comprising an organic active pharmaceutical ingredient thereby preparing inorganic oxide-coated particles comprising an organic active pharmaceutical ingredient enclosed by one or more inorganic oxide layers; and (c) processing the coated particles to prepare a pharmaceutical composition.

In various embodiments: the particles have an aspect ratio of between 5 and 10, 5 and 20, 5 and 50 or greater than 40, the particles consist of a crystalline API or an amorphous API, the particles have a D50 of 0.1 μm to 100 μm on a volume average basis; the particles have a D50 of 0.1 μm to 20 μm on a volume average basis; the particles have a D90 of 0.1 μm to 100 μm on a volume average basis; the particles have a D90 of less than 30 μm on a volume average basis; the particles are dendrites having at least one branch; the particles have an average of at least 3 ends; the uncoated particles have an average of at least 2, 3, 4 or 5 branches; the inorganic oxide coating is 1-10 nm thick on average; the specific surface area of the particles is: greater than 2 m²/g, greater than 4 m²/g, greater than 6 m²/g, greater than 8 m²/g, or between 2 or 4 and 8 m²/g; the flow function coefficient (FFc) of the particles at 3 kPa is between 1 and 6 and the FFc of the coated particles is 1.2 to 10 times the FFc of the uncoated particles; the FFc of the coated particles in at least 1.2, 1.5, 2, 3, 4 or 5 times that of the particles prior to coating; the coated particles are 5%-15% wt/wt inorganic oxide coating; and the organic active pharmaceutical ingredient is an organic compound.

In embodiments: the step of performing atomic layer deposition comprises: (b1) loading the particles comprising the drug into a reactor; (b2) applying a vaporous or gaseous metal or metalloid precursor to the particles in the reactor by pulsing the vaporous or gaseous metal or metalloid precursor into the reactor at least two times; (b3) optionally performing one or more pump-purge cycles of the reactor using inert gas; (b4) applying a vaporous or gaseous oxidant to the particles in the reactor by pulsing the vaporous or gaseous oxidant into the reactor at least two times; and (b5) optionally performing one or more pump-purge cycles of the reactor using inert gas.

In various embodiments: steps (b2)-(b5) are performed two or more times to increase the total thickness of the inorganic oxide layer before step (c) is performed; steps (b2)-(b5) are performed at least four times providing a first, second, third and fourth cycle, and the number of pulses in step (b4) of the fifth and later cycles is less than the number of pulses used in steps (b2) and (b4) of at least one of the first, second, third and fourth cycle; the number of pulses in step (b2) of the fifth cycle is less than an average number of pulses in step (b2) of the first three cycles; the average number of pulses in step (b4) of the first two cycles is greater than an average number of pulses in step (b4) of the last two cycles; the average number of pulses in step (b2) of the first two cycles is greater than an average number of pulses in step (b2) of the remaining cycles; only a portion of vapor or gaseous content is pumped out prior to step (b3) and/or step (b5); the inorganic oxide layer has a thickness in range of 1 nm to 15 nm; step (c) comprises combining the coated particles with one or more pharmaceutically acceptable excipients; the inorganic oxide is selected from the group consisting of: zinc oxide, aluminum oxide, silicon oxide, titanium oxide and combinations thereof; the inorganic oxide is silicon oxide; step (b) takes place at a temperature between 25° C. and 60° C.; the inorganic oxide is selected from the group consisting of aluminum oxide and titanium oxide; the coated particles consist of an organic active pharmaceutical ingredient and an inorganic oxide; the step of further processing the coated particles to create a pharmaceutical composition comprises combining the coated particles with one or more pharmaceutically acceptable excipients; the step of further processing the coated particles to create a pharmaceutical composition comprises combining the coated particles with one or more pharmaceutically acceptable excipients and creating a powder, tablet or capsule or oral suspension or injectable liquid composition.

Also described are: coated particles having a core comprising an organic active pharmaceutical ingredient enclosed by one or more inorganic oxide layers, wherein the coated particles have an aspect ratio greater than 5.

In various embodiments: the one or more inorganic oxide layers have a total thickness in range of 1 nm to 15 nm, 1 nm to 10 nm or 2 nm-10 nm; the core of the coated particles comprises an organic active pharmaceutical ingredient and one or more pharmaceutically acceptable excipients; the inorganic oxide is selected from the group consisting of aluminum oxide, silicon oxide, zinc oxide and titanium oxide and combinations thereof; the coated particles are 5%-15% wt/wt inorganic oxide coating; the active pharmaceutical ingredient is an organic compound; the coated particles have an aspect ratio of between 5 and 100; the core consists of a crystalline organic active pharmaceutical ingredient or an amorphous; the coated particles have a D50 of 0.1 μm to 30 μm on a volume average basis; the coated particles are dendrites; the coated particles are dendrites having an average of at least 3 ends; the coated particles are dendrites having an average of at least 2, 3, 4 or 5 branches; the specific surface area of the coated particles is greater than 2 m²/g, greater than 4 m²/g, greater than 6 m²/g, greater than 8 m²/g, between 2 or 4 and 8 m²/g; the particles have an aspect ratio of 5-50 and a coefficient (FFc) of the particles at 3 kPa is between 1 and 3.

In various embodiments: the coated particles have a bulk density that is at least 5%, 10%, 15%, or 20% greater than the particles prior to coating; the coated particles have a conditioned bulk density that is at least 5%, 10%, 15%, or 20% greater than the particles prior to coating; the coated particles are 2%-10%, 4%-10%, 5%-10% wt/wt inorganic oxide.

Also described is a pharmaceutical composition prepared by any of the forgoing methods and a pharmaceutical composition comprising any of the forgoing coated particles and at least one pharmaceutically acceptable excipient or carrier. In some cases, the uncoated particles are at least 50%, 60%, 65%, 70%, 80%, 90%, 95%, (wt/wt) API; the particles have a D50 of 0.1 μm to 50 μm or 0.1 μm to 20 μm (e.g., 0.1 μm to 10 μm or 0.1 μm to 5 μm, 1 μm to 10 μm or 1 μm to 5 μm or 2 μm to 10 μm or 2 μm to 20 μm) on a volume average basis; the particles have a D90 of 200 μm to 2000 μm on a volume average basis; the coating is 5-100 nm thick, 5-50 nm thick, 10-100 nm thick, 10-50 nm thick or 5-25 nm thick; the coated particles are 1-15%, (e.g., 4%, 6%, 8%, 10%, 12%, 14%, 15%, e.g., 5 or 8 to 15%) wt/wt inorganic oxide; the particles have a specific surface area greater than 0.5 (e.g., greater than 1, 2, 3, 4, 5, 6, 7, 8, 9 or 10) m²/gm (e.g., 1-8 m²/gm)

Prior to coating, the particles may consist of or consist essentially of an API.

The one or more inorganic oxide materials include: aluminum oxide, titanium oxide, iron oxide, gallium oxide, magnesium oxide, zinc oxide, niobium oxide, silicon oxide, hafnium oxide, tantalum oxide, lanthanum oxide, and/or zirconium dioxide.

The oxidant may be selected from the group of water, ozone, and inorganic peroxide.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting.

Other features and advantages of the invention will be apparent from the following detailed description and figures, and from the claims.

### BRIEF DESCRIPTION OF THE DRAWINGS

FIG. 1 is a schematic front view of a reactor for ALD coating of particles, e.g., drugs, that includes a stationary drum.

FIG. 2 is schematic drawing of an example needle-shaped particle that is a dendrite.

FIG. 3 presents scanning electron micrographs of uncoated and coated particles of Compound Alpha (A and B, respectively) and coated and uncoated particles of Compound Beta (C and D, respectively).

FIG. 4 shows cross-sectional TEM images of aluminum oxide (A) and zinc oxide (B) film over the Beta particle surface

FIG. 5 is a graph showing the tensile strength of tablet prepared with coated and uncoated Beta.

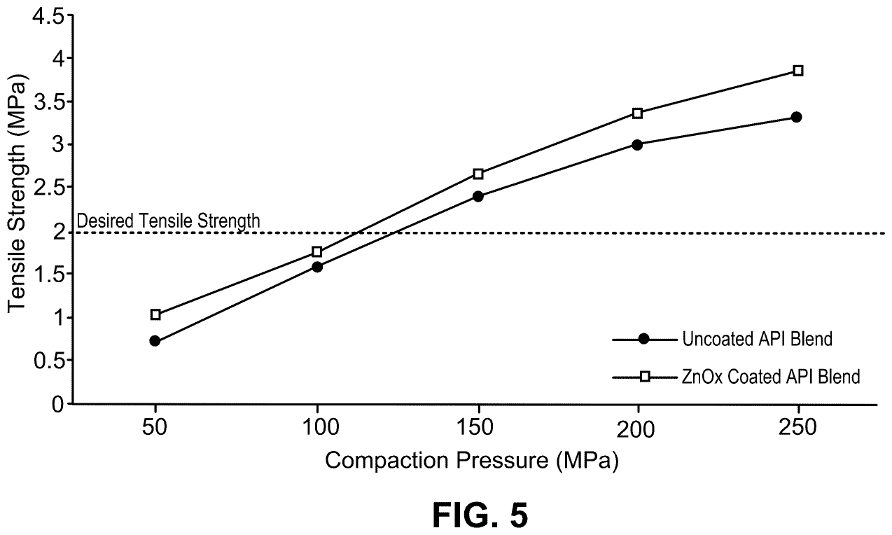

FIG. 6 presents FFc values (left bar of each pair) and CBD values (right bar of each pair) for uncoated cellobiose octaacetate as well as cellobiose octaacetate coated with the indicated weight percent of the aluminum oxide or zinc oxide.

FIG. 7 presents scanning electron micrographs of uncoated and coated particles of cellobiose octaacetate.

### DETAILED DESCRIPTION

The present disclosure provides methods of preparing pharmaceutical compositions comprising particles having a high aspect ratio (i.e., needle-shaped particles) comprising an API coated with one or more layers of an inorganic oxide, e.g., a metal oxide. The coating layers are conformal and the thickness can be controlled using the methods described herein. The coating process described herein can provide particles in which flowability and/or bulk density of the particles is increased. This permits the preparation of needle shaped particles that are easier to handle during the manufacture of a drug product comprising the particles. In addition, because the coating is relatively thin, drug products with high drug loading can be produced. For example, the metal oxide layer can have a thickness in range of 1 nm to 20 nm. In addition, the oxide coating can improve compressibility compared to otherwise identical uncoated particles. In addition, coated particles can have a reduced tendency to agglomerate compared to otherwise identical uncoated particles. Finally, there are benefits with respect to cost and ease of manufacture, for example, because multiple coatings can be applied in the same reactor.

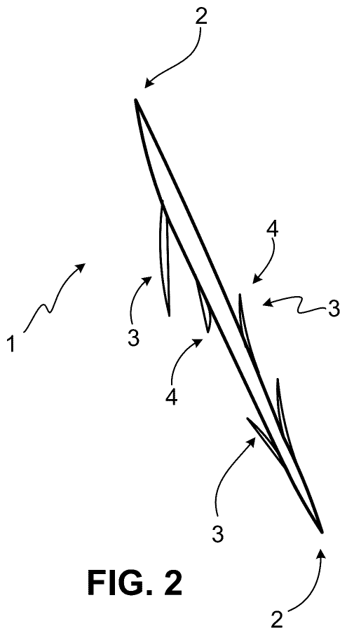

Needle-shaped particles have very poor flowability. Needle-shaped particles that are dendrites (i.e., have branches) have particularly poor flowability due to tangling. Referring to FIG. 2, a needle-shaped drug particle that is a dendrite (1) has two ends (“tips”) (2) and at least one branch (3) each of which has an end (“tip”) (4). Thus, a needle shaped particle with one branch has three ends, the two primary ends and the end of the branch. Each of the branches can themselves be branched (not depicted) and these further branches can be branched. The branches present on needle-shaped particles that are dendrites can cause entangling of the particles, leading to poor flowability and low bulk density.

While a metal oxide coating can improve flowability of particles containing drugs, the improvement of flowability and/or reduction in the tendency to agglomerate that can be achieved by coating needle shaped particles is surprising.

### Drug

The term “drug,” in its broadest sense includes all small molecule (e.g., non-biologic) APIs, in particular APIs that are organic molecules. The drug could be selected from the group consisting of an analgesic, an anesthetic, an anti-inflammatory agent, an anthelmintic, an anti-arrhythmic agent, an antiasthma agent, an antibiotic, an anticancer agent, an anticoagulant, an antidepressant, an antidiabetic agent, an antiepileptic, an antihistamine, an antitussive, an antihypertensive agent, an antimuscarinic agent, an antimycobacterial agent, an antineoplastic agent, an antioxidant agent, an antipyretic, an immunosuppressant, an immunostimulant, an antithyroid agent, an antiviral agent, an anxiolytic sedative, a hypnotic, a neuroleptic, an astringent, a bacteriostatic agent, a beta-adrenoceptor blocking agent, a blood product, a blood substitute, a bronchodilator, a buffering agent, a cardiac inotropic agent, a chemotherapeutic, a contrast media, a corticosteroid, a cough suppressant, an expectorant, a mucolytic, a diuretic, a dopaminergic, an antiparkinsonian agent, a free radical scavenging agent, a growth factor, a haemostatic, an immunological agent, a lipid regulating agent, a muscle relaxant, a parasympathomimetic, a parathyroid calcitonin, a biphosphonate, a prostaglandin, a radio-pharmaceutical, a hormone, a sex hormone, an anti-allergic agent, an appetite stimulant, an anoretic, a steroid, a sympathomimetic, a thyroid agent, a vaccine, a vasodilator and a xanthine.

Exemplary types of small molecule drugs include, but are not limited to, acetaminophen, clarithromycin, azithromycin, ibuprofen, fluticasone propionate, salmeterol, pazopanib HCl, palbociclib, and amoxicillin potassium clavulanate.

### Inorganic or Metal Oxide Material

The term “inorganic oxide material” in its broadest sense includes all materials formed from the reaction of inorganic elements, including metals (e.g., Al or Zn) or metalloids (e.g., Si) with oxygen-containing oxidants. Exemplary metal oxide materials include, but are not limited to, aluminum oxide, titanium dioxide, iron oxide, gallium oxide, magnesium oxide, zinc oxide, niobium oxide, hafnium oxide, tantalum oxide, lanthanum oxide, and zirconium dioxide. Silicon oxide is an example of an inorganic oxide created by reaction between a metalloid and an oxidant. Exemplary oxidants include, but are not limited to, water, ozone, and inorganic peroxide.

### Atomic Layer Deposition (ALD)

The coating layers described are applied by vapor phase deposition using a precursor molecule and an oxidant (e.g., ozone or water vapor). Vapor phase inorganic oxides is sometimes referred to as atomic layer deposition (ALD). However, depending on a number of factors, including the surface being coated, each cycle of the deposition reaction does not necessarily deposit a single atomic layer.

### Reactor System

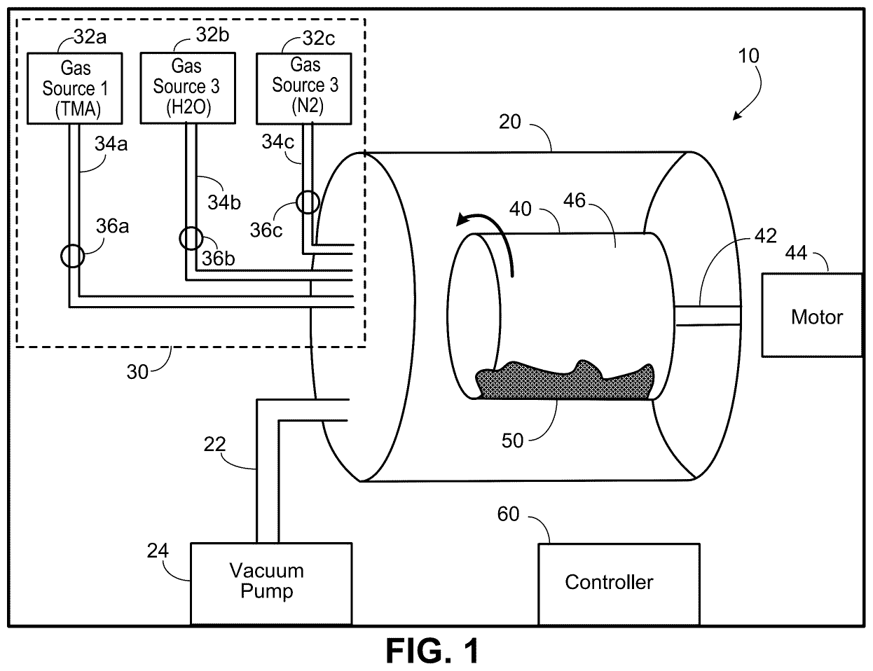

The term “reactor system” in its broadest sense includes all systems that could be used to perform ALD. An exemplary reactor system is illustrated in FIG. 1 and further described below. An alternative reactor system is described in WO 2020/219583.

FIG. 1 illustrates a reactor system 10 for performing coating of particles, with thin-film coatings. The reactor system 10 can perform ALD coating. The reactor system 10 permits ALD coating to be performed at higher (above 50° C., e.g., 50-100° C. or higher) or lower processing temperatures, e.g., below 50° C., e.g., at or below 35° C. For example, the reactor system 10 can form thin-film metal oxides on the particles primarily by ALD at temperatures of 22-35° C., e.g., 25-35° C., 25-30° C., or 30-35° C. In general, the particles can remain or be maintained at such temperatures. This can be achieved by having the reactant gases and/or the interior surfaces of the reactor chamber (e.g., the chamber 20 and drum 40 discussed below) remain or be maintained at such temperatures.

Again, illustrating an ALD process, the reactor system 10 includes a stationary vacuum chamber 20 which is coupled to a vacuum pump 24 by vacuum tubing 22. The vacuum pump 24 can be an industrial vacuum pump sufficient to establish pressures less than 1 Torr, e.g., 1 to 100 mTorr, e.g., 50 mTorr. The vacuum pump 24 permits the chamber 20 to be maintained at a desired pressure and permits removal of reaction byproducts and unreacted process gases.

In operation, the reactor 10 performs the ALD thin-film coating process by introducing gaseous precursors of the coating into the chamber 20. The gaseous precursors are introduced alternatively into the reactor. This permits the ALD process to be a solvent-free process. The half-cycles of the ALD process are self-limiting, which can provide Angstrom level control of deposition. In addition, the ALD reaction can be performed at low temperature conditions, such as below 50° C., e.g., below 35° C.

The chamber 20 is also coupled to a chemical delivery system 30. The chemical delivery system 30 includes three or more gas sources 32a, 32b, 32c coupled by respective delivery tubes 34a, 34b, 34c and controllable valves 36a, 36b, 36c to the vacuum chamber 20. The chemical delivery system 30 can include a combination of restrictors, gas flow controllers, pressure transducers, and ultrasonic flow meters to provide controllable flow rate of the various gasses into the chamber 20. The chemical delivery system 30 can also include one or more temperature control components, e.g., a heat exchanger, resistive heater, heat lamp, etc., to heat or cool the various gasses before they flow into the chamber 20. Although FIG. 1 illustrates separate gas lines extending in parallel to the chamber for each gas source, two or more of the gas lines could be joined, e.g., by one or more three-way valves, before the combined line reaches the chamber 20. In addition, although FIG. 1 illustrates three gas sources, the use of four gas sources could enable the in-situ formation of laminate structures having alternating layers of two different metal oxides.

Two of the gas sources provide two chemically different gaseous reactants for the coating process to the chamber 20. Suitable reactants for ALD methods include any of or a combination of the following: monomer vapor, metal-organics, metal halides, oxidants, such as ozone or water vapor, and polymer or nanoparticle aerosol (dry or wet). For example, the first gas source 32a can provide gaseous trimethylaluminum (TMA) or titanium tetrachloride (TiCl₄), whereas the second gas source 32b can provide water vapor.

One of the gas sources can provide a purge gas. In particular, the third gas source can provide a gas that is chemically inert to the reactants, the coating, and the particles being processed. For example, the purge gas can be N₂, or a noble gas, such as argon.

A rotatable coating drum 40 is held inside the chamber 20. The drum 40 can be connected by a drive shaft 42 that extends through a sealed port in a side wall of the chamber 20 to a motor 44. The motor 44 can rotate the drum at speeds of 1 to 100 rpm. Alternatively, the drum can be directly connected to a vacuum source through a rotary union.

The particles to be coated, shown as a particle bed 50, are placed in an interior volume 46 of the drum 40. The drum 40 and chamber 20 can include sealable ports (not illustrated) to permit the particles to be placed into and removed from the drum 40.

The body of the drum 40 is provided by one or more of a porous material, a solid metal, and a perforated metal. The pores through the cylindrical side walls of the drum 40 can have a dimension of 10 μm.

In operation, one of the gasses flows into chamber 20 from the chemical delivery system 30 as the drum 40 rotates. A combination of pores (1-100 um), holes (0.1-10 mm), or large openings in the coating drum 40 serve to confine the particles in the coating drum 40 while allowing rapid delivery of precursor chemistry and pumping of byproducts or unreacted species. Due to the pores in the drum 40, the gas can flow between the exterior of the drum 40, i.e., the reactor chamber 20, and the interior of the drum 40. In addition, rotation of the drum 40 agitates the particles to keep them separate, ensuring a large surface area of the particles remains exposed. This permits fast, uniform interaction of the particle surface with the process gas.

In some implementations, one or more temperature control components are integrated into the drum 40 to permit control of the temperature of the drum 40. For example, resistive heater, a thermoelectric cooler, or other component can be integrated in or on the side walls of the drum 40.

The reactor system 10 also includes a controller 60 coupled to the various controllable components, e.g., vacuum pump 24, chemical delivery or gas distribution system 30, motor 44, a temperature control system, etc., to control operation of the reactor system 10. The controller 60 can also be coupled to various sensors, e.g., pressure sensors, flow meters, etc., to provide closed loop control of the pressure of the gasses in the chamber 20.

In general, the controller 60 can operate the reactor system 10 in accord with a “recipe.” The recipe specifies an operating value for each controllable element as a function of time. For example, the recipe can specify the times during which the vacuum pump 24 is to operate, the times of and flow rate for each gas source 32a, 32b, 32c, the rotation rate of the motor 44 or drum 40, etc. The controller 60 can receive the recipe as computer-readable data (e.g., that is stored on a non-transitory computer readable medium).

The controller 60 and other computing devices part of systems described herein can be implemented in digital electronic circuitry, or in computer software, firmware, or hardware. For example, the controller can include a processor to execute a computer program as stored in a computer program product, e.g., in a non-transitory machine-readable storage medium. Such a computer program (also known as a program, software, software application, or code) can be written in any form of programming language, including compiled or interpreted languages, and it can be deployed in any form, including as a standalone program or as a module, component, subroutine, or other unit suitable for use in a computing environment. In some implementations, the controller 60 is a general-purpose programmable computer. In some implementations, the controller can be implemented using special purpose logic circuitry, e.g., an FPGA (field programmable gate array) or an ASIC (application specific integrated circuit).

### Operation

Initially, particles are loaded into the drum 40 in the reactor system 10. The particles can have a solid core comprising a drug, e.g., one of the drugs discussed above. Once any access ports are sealed, the controller 60 operates the reactor system 10 according to the recipe in order to form the thin-film metal oxide layers on the particles.

In particular, the two reactant gases are alternately supplied to the chamber 20, with each step of supplying a reactant gas followed by a purge cycle in which the inert gas is supplied to the chamber 20 to force out the reactant gas and by-products used in the prior step. Moreover, one or more of the gases (e.g., the reactant gases and/or the inert gas) can be supplied in pulses in which the chamber 20 is filled with the gas to a specified pressure, a delay time is permitted to pass, and the chamber is evacuated by the vacuum pump 24 before the next pulse commences.

In particular, the controller 60 can operate the reactor system 10 as follows.

In a first reactant cycle (called a half-cycle), while the motor 44 rotates the drum 40 to agitate the particles 50:
i) The gas distribution system 30 is operated to flow the first reactant gas, e.g., TMA, from the source 32a into the chamber 20 until a first specified pressure is achieved. The specified pressure can be 0.1 Torr to half of the saturation pressure of the reactant gas.
ii) Flow of the first reactant is halted, and a specified delay time is permitted to pass, e.g., as measured by a timer in the controller. This permits the first reactant to flow through the particle bed in the drum 40 and react with the surface of the particles 50 inside the drum 40.
iii) The vacuum pump 50 evacuates the chamber 20, e.g., down to pressures below 1 Torr, e.g., to 1 to 100 mTorr, e.g., 50 mTorr.

These steps (i)-(iii) can be repeated a number of times set by the recipe, e.g., two to ten times, e.g., six times.

Next, in a first purge cycle, while the motor 44 rotates the drum to agitate the particles 50:
iv) The gas distribution system 30 is operated to flow the inert gas, e.g., N₂, from the source 32c into the chamber 20 until a second specified pressure is achieved. The second specified pressure can be 1 to 100 Torr.
v) Flow of the inert gas is halted, and a specified delay time is permitted to pass, e.g., as measured by the timer in the controller. This permits the inert gas to flow through the pores in the drum 40 and diffuse through the particles 50 to displace the reactant gas and any vaporous by-products.
vi) The vacuum pump 50 evacuates the chamber 20, e.g., down to pressures below 1 Torr, e.g., to 1 to 500 mTorr, e.g., 50 mTorr.

These steps (iv)-(vi) can be repeated a number of times set by the recipe, e.g., six to twenty times, e.g., sixteen times. Taken together steps (iv)-(vi) are called a pump-purge cycle.

In a second reactant half-cycle, while the motor 44 rotates the drum 40 to agitate the particles 50:
vii) The gas distribution system 30 is operated to flow the second reactant gas, e.g., H2O, from the source 32b into the chamber 20 until a third specified pressure is achieved. The third pressure can be 0.1 Torr to half of the saturation pressure of the reactant gas.
viii) Flow of the second reactant is halted, and a specified delay time is permitted to pass, e.g., as measured by the timer in the controller. This permits the second reactant to flow through the pores in the drum 40 and react with the surface of the particles 50 inside the drum 40.
ix) The vacuum pump 50 evacuates the chamber 20, e.g., down to pressures below 1 Torr, e.g., to 1 to 500 mTorr, e.g., 50 mTorr.

These steps (vii)-(ix) can be repeated a number of times set by the recipe, e.g., two to ten times, e.g., six times.

Next, a second purge cycle is performed. This second purge cycle can be identical to the first purge cycle, or can have a different number of repetitions of the steps (iv)-(vi) and/or different delay time and/or different pressure.

The cycle of the first reactant half-cycle, first purge cycle, second reactant half cycle and second purge cycle can be repeated a number of times set by the recipe, e.g., one to ten times.

As noted above, the coating process can be performed at a low processing temperature, e.g., below 50° C., e.g., at or below 35° C. In particular, the particles can remain or be maintained at such temperatures during all of steps (i)-(ix) noted above. In general, the temperature of the interior of the reactor chamber does not exceed 35° C. during of steps (i)-(ix). This can be achieved by having the first reactant gas, second reactant gas and inert gas be injected into the chamber at such temperatures during the respective cycles. In addition, physical components of the chamber can remain or be maintained at such temperatures, e.g., using a cooling system, e.g., a thermoelectric cooler, if necessary.

### Process for Preparing Pharmaceutical Compositions Comprising Drugs Encapsulated by One or More Layers of Inorganic Oxide

Provided are two exemplary methods for a pharmaceutical composition comprising an API-containing core (a needle-shaped particle comprising an API) enclosed by one or more layers of an inorganic oxide (e.g., a metal oxide). The first exemplary method includes the sequential steps of: (a) loading the particles comprising the API into a reactor and evacuating the reactor; (b) applying a vaporous or gaseous inorganic or metal precursor to the particles in the reactor (in some embodiments comprising at least two pulses); (c) performing one or more pump-purge cycles of the reactor using inert gas; (d) applying a vaporous or gaseous oxidant to the particles in the reactor (e.g., comprising at least two pulses); and (e) performing one or more pump-purge cycles of the reactor using inert gas. In some embodiments of the first exemplary method, the sequential steps (b)-(e) are optionally repeated one or more times to increase the total thickness of the one or more inorganic or metal oxide materials that enclose the particles. In some embodiments, the reactor pressure is allowed to stabilize following step (a), step (b), and/or step (d). In some embodiments, the reactor contents are agitated prior to and/or during step (b), step (c), and/or step (e). In some embodiments, a portion of vapor or gaseous content is pumped out prior to step (c) and/or step (e).

The second exemplary method includes (e.g., consists of) the sequential steps of (a) loading the particles comprising a drug (e.g., particles of a drug and one more excipients) into a reactor, (b) reducing the reactor pressure to less than 1 Torr, (c) agitating the reactor contents until the reactor contents have a desired moisture content, (d) pressurizing the reactor to at least 10 Torr by adding a vaporous or gaseous inorganic or metal precursor (e.g., comprising at least two pulses), (e) allowing the reactor pressure to stabilize, (f) agitating the reactor contents, (g) pumping out a portion of vapor or gaseous content, and determining when to stop pumping based on analysis of content in the reactor including an inorganic or metal precursor and a byproduct of an inorganic metal precursor, (h) performing a sequence of pump-purge cycles of the reactor using insert gas, (i) pressuring the reactor to at least 10 Torr by adding a vaporous or gaseous oxidant (e.g., comprising at least two pulses), (j) allowing the reactor pressure to stabilize, (k) agitating the reactor contents, (I) pumping out a portion of vapor or gaseous content and determining when to stop pumping based on analysis of content in the reactor including the metal precursor, the byproduct of the metal precursor reacting with exposed hydroxyl residues on the substrate or on the particle surface, and unreacted oxidant, and (m) performing a sequence of pump-purge cycles of the reactor using insert gas. In some embodiments of the second exemplary method, the sequential steps (b)-(m) are optionally repeated one or more times to increase the total thickness of the one or more metal oxide materials that enclose the particles.

Some embodiments provide a method of preparing a pharmaceutical composition comprising coated needle-shaped particles comprising an active pharmaceutical ingredient enclosed by one or more inorganic or metal oxide layers, the method comprising the sequential steps of: (a) providing uncoated needle-shaped particles comprising an active pharmaceutical ingredient (API); (b) performing atomic layer deposition to apply a metal oxide layer to uncoated needle-shaped particles comprising an active pharmaceutical ingredient thereby preparing coated particles comprising an active pharmaceutical ingredient enclosed by one or more metal oxide layers; (c) processing the coated particles to prepare a pharmaceutical composition wherein the processing comprising combining the particles with one or more pharmaceutically acceptable (e.g., acceptable in an oral drug product) excipients; and (d) processing the pharmaceutical composition to form a drug product (e.g., a pill, tablet, liquid, suspension or capsule). In some cases, the drug product is an oral drug product.

In some embodiments, the uncoated needle-shaped particles are at least 50% wt/wt API. In some embodiments, the uncoated particles are at least 70%, 80%, 90%, 99% or 100% wt/wt API. In some cases, the API is crystalline. In some cases, the API is amorphous.

In some embodiments, the step of performing atomic layer deposition comprises: (b1) loading the particles comprising the drug into a reactor; (b2) applying a vaporous or gaseous metal precursor to the particles in the reactor; (b3) performing one or more pump-purge cycles of the reactor using inert gas; (b4) applying a vaporous or gaseous oxidant to the particles in the reactor; and (b5) performing one or more pump-purge cycles of the reactor using inert gas. In some embodiments, steps (b2)-(b5) are performed two or more times to increase the total thickness of the metal oxide layer before step (c) is performed.

In some embodiments, the reactor pressure is allowed to stabilize following step (b1), step (b2), and/or step (b4). In some embodiments, the reactor contents are agitated prior to and/or during step (b1), step (b3), and/or step (b5). In some embodiments, a subset of vapor or gaseous content is pumped out prior to step (b3) and/or step (b5). In some embodiments, step (b) takes place at a temperature between 35° C. and 50° C. In some embodiments, step (c) comprises combining the coated particles with one or more pharmaceutically acceptable excipients.

In some embodiments, the metal oxide layer has a thickness in range of 0.1 nm to 100 nm.

In some embodiments, the metal oxide is selected from the group consisting of: zinc oxide, aluminum oxide, silicon oxide and titanium oxide. In some embodiments, the metal oxide is aluminum oxide. In some embodiments, the metal oxide is selected from the group consisting of aluminum oxide and titanium oxide.

Some embodiments provide a pharmaceutical composition comprising coated needle-shaped particles comprising an active pharmaceutical ingredient enclosed by one or more metal oxide layers, prepared by a method comprising the sequential steps of: (a) providing uncoated needle-shaped particles comprising an active pharmaceutical ingredient; (b) performing atomic layer deposition to apply a metal oxide layer to uncoated needle-shaped particles comprising an active pharmaceutical ingredient thereby preparing coated needle-shaped particles comprising an active pharmaceutical ingredient enclosed by one or more metal oxide layers; and (c) processing the coated needle-shaped particles to prepare a pharmaceutical composition.

In some embodiments, the step of performing atomic layer deposition comprises:
(b1) loading the particles comprising the drug into a reactor; (b2) applying a vaporous or gaseous metal precursor to the particles in the reactor; (b3) performing one or more pump-purge cycles of the reactor using inert gas; (b4) applying a vaporous or gaseous oxidant to the particles in the reactor; and (b5) performing one or more pump-purge cycles of the reactor using inert gas.

In some embodiments, steps (b2)-(b5) are performed two or more times to increase the total thickness of the metal oxide layer before step (c) is performed. In some embodiments, the particles are agitated during step (b). In some embodiments, the reactor pressure is allowed to stabilize following step (b1), step (b2), and/or step (b4). In some embodiments, the reactor contents are agitated prior to and/or during step (b1), step (b3), and/or step (b5). In some embodiments, a subset of vapor or gaseous content is pumped out prior to step (b3) and/or step (b5). In some embodiments, step (b) takes place at a temperature between 35° C. and 50° C.

In some embodiments, the metal oxide layer has a thickness in range of 1 nm to 5, 10 or 15 nm. In some embodiments, the uncoated particles have a median particle size on a volume average basis between 0.1 μm and 1000 μm.

In some embodiments, the coated particles comprising an active pharmaceutical ingredient further comprise one or more pharmaceutically acceptable excipients. In some embodiments, the uncoated particles consist of the active pharmaceutical ingredient.

For example, a method for creating an aluminum oxide coating can include the steps of:
(a) loading particles comprising the drug into a reactor;
(b) reducing the reactor pressure to less than 1 Torr;
(c) agitating the reactor contents until the reactor contents has a desired water content by performing residual gas analysis (RGA) to monitor levels of water vapor in the reactor;
(d) pressurizing the reactor to at least 1 Torr by adding a vaporous or gaseous TMA;
(e) allowing the reactor pressure to stabilize;
(f) agitating the reactor contents;
(g) pumping out a subset of vapor or gaseous content, including gaseous methane and unreacted TMA, and determining when to stop pumping by performing RGA to monitor levels of gaseous methane and unreacted TMA in the reactor;
(h) performing a sequence of pump-purge cycles on the reactor using nitrogen gas;
(i) pressuring the reactor to at least 1 Torr by adding water vapor;
(j) allowing the reactor pressure to stabilize;
(k) agitating the reactor contents;
(l) pumping out a subset of vapor or gaseous content, including water vapor, and determining when to stop pumping by performing RGA to monitor levels of water vapor in the reactor; and
(m) performing a sequence of pump-purge cycles on the reactor using nitrogen gas.

In some cases, the steps of (b)-(m) are repeated more than once to increase the total thickness of the metal oxide that encloses the drug particle core. A zinc oxide coating can be applied by a similar process using diethyl zinc (CH₃CH₂)₂Zn. A titanium oxide coating can be applied by a similar process using TiCl₄.

For particles with a high specific surface area and/or high aspect ratio, it is desirable for the number of pulses in each half cycle to be higher than that generally used for particles with a lower specific surface area and/or low aspect ratio (with the same mass loading). However, in many cases, the number of pulses in each half cycle can be decreased in later cycles (when the thickness of the coating is greater) even for particles with a high specific surface area and/or high aspect ratio. In addition, it is generally expected for the weight percent of the coating material in the coated particle to be higher for particles with a higher specific surface area and/or aspect ratio than those with a lower specific surface area and/or aspect ratio.

### Pharmaceutically Acceptable Excipients, Diluents, and Carriers

Pharmaceutically acceptable excipients include, but are not limited to:
(1) surfactants and polymers including: polyethylene glycol (PEG), polyvinylpyrrolidone (PVP), sodium lauryl sulfate, polyvinylalcohol, crospovidone, polyvinylpyrrolidone-polyvinylacrylate copolymer (PVPVA), cellulose derivatives, hydroxypropylmethyl cellulose, hydroxypropyl cellulose, carboxymethylethyl cellulose, hydroxypropyllmethyl cellulose phthalate, polyacrylates and polymethacrylates, urea, sugars, polyols, carbomer and their polymers, emulsifiers, sugar gum, starch, organic acids and their salts,
(2) binding agents such as cellulose, cross-linked polyvinylpyrrolidone, microcrystalline cellulose;
(3) filling agents such as lactose monohydrate, lactose anhydrous, microcrystalline cellulose and various starches;
(4) lubricating agents such as agents that act on the flowability of a powder to be compressed, including colloidal silicon dioxide, talc, stearic acid, magnesium stearate, calcium stearate, silica gel;
(5) sweeteners such as any natural or artificial sweetener including sucrose, xylitol, sodium saccharin, cyclamate, aspartame, and acesulfame K;
(6) flavoring agents;
(7) preservatives such as potassium sorbate, methylparaben, propylparaben, benzoic acid and its salts, other esters of parahydroxybenzoic acid such as butylparaben, alcohols such as ethyl or benzyl alcohol, phenolic chemicals such as phenol, or quarternary compounds such as benzalkonium chloride;
(8) buffers;
(9) diluents such as pharmaceutically acceptable inert fillers, such as microcrystalline cellulose, lactose, dibasic calcium phosphate, saccharides, and/or mixtures of any of the foregoing;
(10) wetting agents such as corn starch, potato starch, maize starch, and modified starches, and mixtures thereof;
(11) disintegrants; such as croscarmellose sodium, crospovidone, sodium starch glycolate; and
(12) effervescent agents such as effervescent couples such as an organic acid (e.g., citric, tartaric, malic, fumaric, adipic, succinic, and alginic acids and anhydrides and acid salts), or a carbonate (e.g., sodium carbonate, potassium carbonate, magnesium carbonate, sodium glycine carbonate, L-lysine carbonate, and arginine carbonate) or bicarbonate (e.g. sodium bicarbonate or potassium bicarbonate).

### EXAMPLES

### Materials and Methods

The following materials and methods were used in Example 1. Similar methods were used for Example 2.

### APIs

In Example 1, the coated APIs were: Compound Alpha and Compound Beta. Both APIs have poor powder flow characteristics, challenging morphology, different particle size distributions and different specific surface areas and possess a variety of functional groups (phenolic, amide, xanthine derivatives etc.). In Example 2, the API coated was cellobiose octaacetate.

### Atomic Layer Deposition (ALD) Process

The various APIs were coated with aluminum oxide or zinc oxide by ALD essentially as described above, but with a rotating drum or rotating paddle reactor. The processes were carried out at low temperatures to ensure the stability of the APIs during the coating process.

### Coating and Particle Characterization

### Coating Conformality & Coating Compositional Analysis

Oxide coating microstructure, thickness, and conformality were examined by Transmission Electron Microscopy (TEM) and Scanning Electron Microscopy (SEM). Sample particles were encapsulated in epoxy and then cross-sectioned by focused ion beam (FIB) on a FEI Helios NanoLab 600i DualBeam—FIB/SEM instrument (FEI Company (Thermo Fisher Scientific), Hillsboro, OR USA). TEM imaging was performed on a FEI CM200 transmission electron microscope (FEI Company (Thermo Fisher Scientific), Hillsboro, OR USA) at 200 kV.

### Morphology of Particles

Particle morphology before and after coating was examined using SEM. Powder samples were dispersed onto conductive tapes on a SEM sample holder and coated with a thin layer of metal coating. SEM images were taken at 10 kV.

### Specific Surface Area and Particle Size Distribution

Specific surface area (SSA) of powder samples was measured using the standard 5 points BET surface area method using a Micromeritics Gemini VII surface area analyzer (Micromeritics Instrument Corp, Norcross, GA USA). About one gram of powder was degassed at 35° C. (for ibuprofen, considering its melting point) and 60° C. (for other APIs) in flowing nitrogen overnight. The nitrogen adsorption isotherm at relative pressures from 0.05 to 0.25 was collected at liquid nitrogen temperature (77 K) and SSA calculated according to BET theory. When the sample was not sufficient, a single measurement was carried out. Otherwise, multiple measurements were performed.

### Particle Size Distribution

Particle size and distribution of model APIs were analyzed by laser diffraction with Malvern MasterSizer 3000 (Malvern Panalytical Ltd, Malvern, United Kingdom). Powder samples were dispersed by dry dispersion at a dispersing air pressure of 3 bar. Particle size and size distribution of Compounds Alpha and Beta were analyzed by static imaging analysis using a Malvern Morphologi 4 (Malvern Panalytical Ltd, Malvern, United Kingdom). Samples were dispersed onto a glass slide using the sample dispersing unit (SDU) at a dispersing air pressure of 3 bar. Appropriate objective lens is verified and used for the automated imaging analysis. A circular equivalent diameter is used for particle size. When sample was not sufficient, a single measurement was carried out. Otherwise, multiple measurements were performed.

### Crystal Form Analysis

Changes in crystal phases (polymorphs) were evaluated by X-Ray powder diffractometry (XRD). XRD was performed on a Rigaku SmartLab diffractometer (Rigaku, Tokyo, Japan) with Cu kα (λ=1.54184 Å) radiation at 45 kV and 200 mA X-Ray source setting at room temperature. XRD data was collected in the 2θ range of 3-45° (2θ range varies depending on the material), with a step size of 0.02°. Powder sample was packed onto a glass sample holder with a 0.5 mm deep recess of size 20 mm×20 mm to obtain a smooth surface for diffractometry.

### Chemical Composition Analysis

Fourier transform infrared spectroscopy (FTIR) was used to analyze the materials for any changes in chemical composition on a Thermo Nicolet Nexus 870 FTIR spectrometer (Thermo Fisher Scientific, Waltham, MA USA). KBr pellet method was used in transmission mode FTIR. 100 mg powder mixture containing 1 wt % API sample in KBr powder was mixed thoroughly using a mortar and pestle. The mixture was then pressed into a pellet of 13 mm diameter at 15 MPa normal load to achieve a transparent pellet for FTIR measurement. Spectra were collected at the wavenumber range of 400-4000 cm⁻¹ at 4 cm⁻¹ resolution.

### Powder Rheology

Flow properties of powder samples were evaluated using 25 mm vessels on a Freeman Technology FT4 powder rheometer (Freeman Technology (Micromeritics), Tewkesbury, United Kingdom) for the following properties:
Bulk density (BD) from stability and variable flow rate test;
Compressibility at 15 kPa from compressibility test; and
Flow function coefficient (FFc) from shear cell test at 3 kPa and 6 kPa pre-consolidation stress.

The standard test procedure was used, where the powder bed was initially conditioned with the rotating blade which gently sliced the powder bed surface to homogenize the bulk density of powder before testing. A vented piston was used to compact the powders with the desired compaction load. The cell is then split to remove any material above a bed height. Then the rotational shear cell was used to measure the flow function values.

Flow function values (ffc) at specific consolidation stress was calculated using the following equation

$$f f c = \frac{σ 1}{σ c}$$

where σ1 is the major principal stress, and ac is the unconfined yield strength. Both of these were derived from a mathematical treatment (Mohr's circles) of the experimental stress data (incipient shear stresses at each normal stress level).

### Metal Oxide Content and Thickness

Thermogravimetric analysis (TGA) was used to measure oxide coating content for these samples. TGA measurements were performed on a TA Instruments TGA Q50 (TA Instruments (Waters), DE, USA). Samples of 20-30 mg were burned in a dry air environment at a rate of 10° C./min to 800° C., and the residual weight was used to calculate the coating weight content.

The TGA technique was verified on selected samples by measuring metal content with Inductively Coupled Plasma Optical Emission Spectroscopy (ICP-OES) using a Perkin Elmer Optima 7300V (Perkin Elmer, Waltham, MA, USA). Samples were first ashed to burn off any organic material and then digested in a microwave digestion system using a combination of nitric, hydrochloric, and hydrofluoric, acids prior to analysis and spike recovery tests were used to verify the instrument's ability to detect elements of interest. Coating thicknesses were estimated from TGA and ICP-OES results using specific surface and assumed film densities and stoichiometry.

### Blending and Compression Study

Blending: Beta along with colloidal silicon oxide (Ca-O-Sil M-5P; Cabot; Boston, MA, USA), microcrystalline cellulose (Vivapur PH102; JRS, Patterson, NJ, USA), lactose (Spray Dried Fast Flo 316; Kerry; Westport, CT), Colloidal silicon dioxide (Cab-O-Sil M-5P; Cabot; Boston, MA) and croscarmellose sodium (Viva Sol GF; JRS; Patterson, NY), were screened through US sieve #20. The screened ingredients were loaded in a 0.5 qt V-blender and mixed at 30 rpm for 10 minutes. Magnesium stearate (Grade 2257-24; Mallinckrodt; St. Louis, MO, USA) was screened through US sieve #40 and added to the blend and mixed at 30 rpm for 2 minutes. The blend so produced was characterized for appearance, FFc, bulk density, tap density and true density.

Tableting: The blends prepared above were compressed using a single station manual tablet compression machine (MTCM-I, Globe Pharma; New Brunswick, NJ, USA) using 8 mm standard convex tooling. Each blend was compressed at compaction pressures of 50, 100, 150, 200 and 250 MPa. Tablets so prepared were characterized for weight, thickness, and breaking force.

### Example 1: Metal Oxide Coated Particles with High Aspect Ratio Exhibit Increased Flowability and Higher Conditioned Bulk Density Compared to Uncoated Particles

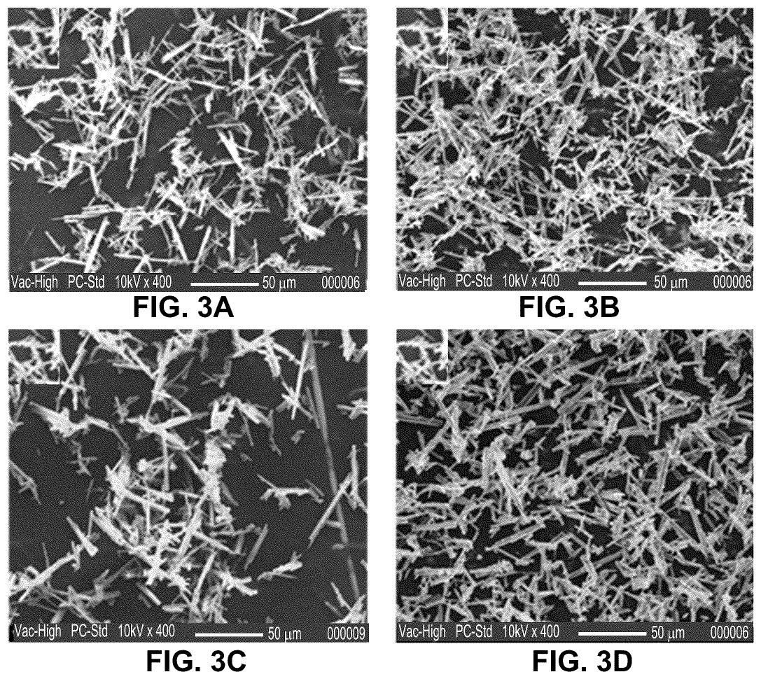

Table 1 provides data regarding the particle characteristics of Compounds Alpha and Beta. It can be seen that they both have a high specific surface area, as is typical for a needle-shaped particle. These needle-shaped APIs were highly cohesive and extremely challenging to handle. SEM images of uncoated Alpha and Beta are provided in FIG. 3 (A and C, respectively).

TABLE 1
Physical characteristics of Alpha and Beta

| | SSA | Bulk | Tap | | | |
|---|---|---|---|---|---|---|
| Material | (m2/g) | density | density | D10 (μm) | D50 (μm) | D90 (μm) |
| Alpha | 4.6 | 0.099 | 0.179 | 7.5 ± 0.5 | 17.5 ± 2.1 | 28.4 ± 4.9 |
| Beta | 2.6 | 0.152 | 0.301 | 8.4 ± 0.5 | 16.3 ± 0.8 | 27.0 ± 1.2 |

Alpha and Beta were coated with zinc oxide by ALD. SEM images of coated Alpha and Beta are shown in FIG. 3 (B and D, respectively). It can be seen that for both APIs, coating with zinc oxide had no effect on the size and the morphology of the particles.

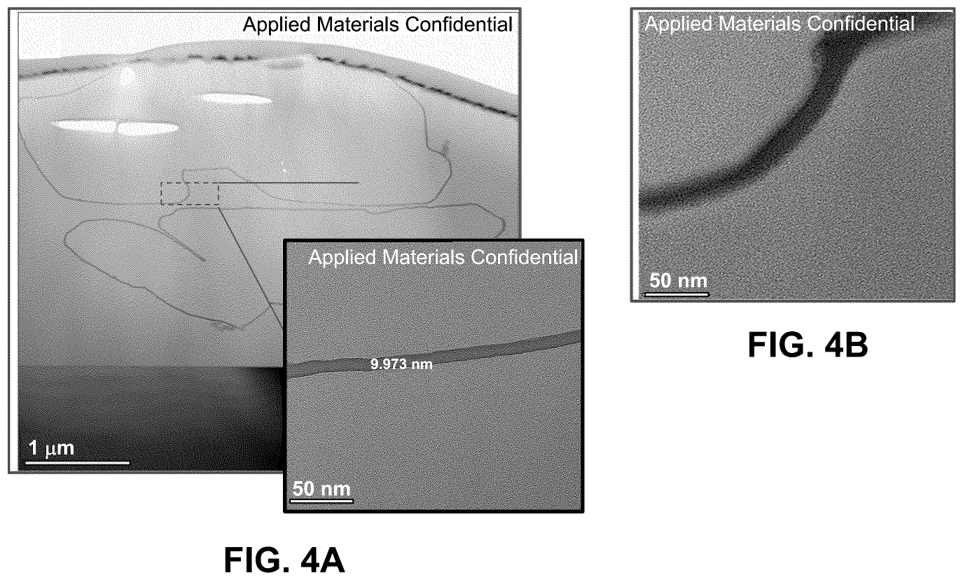

FIG. 4 present cross-sectional TEM images of zinc oxide film over the Beta particle surface. From the low magnification image (FIG. 3A), zinc oxide coating around the particle surface is clearly visible. It can be seen that the coating is highly conformal and uniformly coats the particles all around the surface including the interparticle gaps, pores and sharp corners.

Table 2 provides the powder flow characteristics as measured by bulk density and flow function coefficient (FFc at 3 kPa pre-consolidation force) for uncoated and coated Alpha and Beta. The oxide content of coated Alpha particles is higher than coated Beta particles, because of the higher surface area of Alpha particles. Thus, for the same thickness, the coating wt % required by Alpha particles was higher than that for Beta particles. For coated Alpha particles, the oxide content varied from 4.6 to 14.4% whereas for the coated Beta particles it ranged from 1.5 to 3.7%.

Both uncoated Alpha particles and uncoated Beta particles had a very low bulk density (BD) of 0.100 and 0.130 g/cm³ respectively. The zinc oxide coating substantially improved the bulk density for both APIs. Based on the FFc values, uncoated Alpha particles were categorized as very cohesive (1<FFc<2), and Beta particles were easy flowing. ALD coating with zinc oxide significantly improved the flowability of the Alpha particles as supported by the FFc values provided in Table 2. After zinc oxide coating, the very cohesive Alpha particles turned into easy flowing powder (4<FFc<10). For Beta particles, zinc oxide coating improved flowability grade from easy flowing to free-flowing powder. Thus, for both these APIs, a zinc oxide coating resulted in significant improvement in bulk density and FFc.

TABLE 2
Bulk density and flow function coefficient (FFc) as a
function of zinc oxide coating wt % on Alpha and Beta

| | Oxide | | | |
|---|---|---|---|---|
| | Content | BD | FFc | Flowability |
| Sample | (wt %) | (g/cm³) | (at 3 kPa) | grade |
| Uncoated Alpha | NA | 0.10 | 1.9 ± 0.0* | Very cohesive |
| Zinc oxide | 4.6 | 0.12 | 4.1 ± 0.1 | Easy flowing |
| coated Alpha | 8.8 | 0.13 | 5.4 ± 0.7 | |
| | 14.4 | 0.20 | 6.5 ± 2.2 | |
| Uncoated Beta | NA | 0.13 | 5.4 ± 1.8 | Easy flowing |
| Zinc oxide | 1.6 | 0.18 | 5.7 ± 1.2 | |
| coated Beta | 2.6 | 0.22 | 9.8 ± 3.3 | |
| | 3.7 | 0.21 | 13.0 ± 2.8 | Free flowing |

*Note:
For FFc, samples were studied for replicates or triplicates based on material availability. BD measurements were conducted only once due to limited availability of the samples.

Table 2 also shows the effect of increase in coating oxide content (thickness) on the BD and FFc values. The flowability and bulk density of the APIs improved with the increase in zinc oxide content. For Alpha, BD increased linearly from 0.10 g/cm³ to 0.20 g/cm³ with increase in coating content. Same trend is observed for Beta, where BD improved from 0.13 g/cm³ to 0.21 g/cm³. FFc for both APIs, increased linearly with increase in oxide content. For Alpha, coating reduced the cohesion, and the powder became easy flowing after coating with ˜5 wt % of zinc oxide and it improved further with higher coating thickness. For Beta, FFc increased significantly with the oxide content at >2.6% wt % coating where the easy flowing powder became free flowing.

During pharmaceutical product development and manufacture, the ability to modulate powder flowability can be of immense value. For example, a very cohesive material with poor flow properties may result in non-uniform blending. On the other hand, a free-flowing material with very low cohesion, may cause de-mixing and segregation in the blend or impact tensile strength in the finished tablet. A benefit of ALD coating is the ability to tailor process conditions and coating thickness to achieve a desired set of powder flow properties.

To demonstrate the benefits of ALD on the processability of final dosage form, direct compaction blend with 25% drug load and commonly used excipients were prepared for Beta particles. The quantitative compositions of the blends are described in Table 3. Four blends were prepared using (a) uncoated Beta (Blend A and B) and (b) zinc oxide coated Beta (2.6 wt. %, Blends C and D). The blends comprised of 25% of API-2 (uncoated or coated), Avicel PH 101 as filler/binder, Fast Flo Lactose 316 as filler, Croscarmellose Sodium as disintegrant, Colloidal SiO₂ as glidant (for Blends B and D) and magnesium stearate as lubricant. Additionally, the powder flow improvement achieved due to ALD coating to that obtained through traditional pharmaceutical approach of using glidant was also compared.

TABLE 3
Composition of the direct compaction tableting
blend formulation used in the study.

| | | | Zinc oxide | Zinc oxide |
|---|---|---|---|---|
| | | | coated | Coated |
| | Uncoated | Uncoated | (2.6 wt %) | (2.6 wt %) |
| Blend Ingredient | Blend A | Blend B | Blend C | Blend D |
| Beta | 25 | 25 | 25 | 25 |
| Avicel PH 102 | 46 | 45 | 46 | 45 |
| Fast Flo Lactose | 23 | 23 | 23 | 23 |
| 316 | | | | |
| Croscarmellose | 5 | 5 | 5 | 5 |
| Sodium | | | | |
| Colloidal SiO₂ | 0 | 1 | 0 | 1 |
| Mg Stearate | 1 | 1 | 1 | 1 |
| Total | 100 | 100 | 100 | 100 |

Improvement in bulk density, tap density, compressibility index and conditional bulk density was observed for the coated blends compared to uncoated blends (Blend A vs Blend C; Blend B vs Blend D) as shown in Table 4. Zinc oxide coated API blends (Blend C and D) showed significant improvement to the blend flowability and Blend D resulted into free-flowing grade. These trends were similar to the trends observed for the flow behavior of the uncoated and zinc oxide coated API. The flowability of the blend is critical to achieve the improved process (flowing through hopper, feeder performance, mixing efficiency, filling the die, etc.) and consistent quality tablets with acceptable critical quality attributes such as uniformity of dosage, assay, and dissolution. Thus, this study demonstrated that the improvement in processing properties for the API are directly transferred to corresponding improvement in the final blend properties, resulting in the suitability of the blends for easy processing technologies like direct compression.

TABLE 4
Summary of properties of Beta blends prepared (25% drug load).

| | Uncoated | | Zinc oxide coated | |
|---|---|---|---|---|
| | Beta | | Beta | |
| | blends | | blends | |
| Property | Blend A | Blend B | Blend C | Blend D |
| Appearance | Off-white | Off-white | Off-white | Off-White |
| | color | color | color | color |
| | blend | blend | blend | blend |
| Bulk Density | 0.264 | 0.254 | 0.362 | 0.392 |
| (g/cm³) | | | | |
| Tapped Density | 0.508 | 0.470 | 0.614 | 0.594 |
| (g/cm³) | | | | |
| Compressibility | 48 | 46 | 41 | 34 |
| Index (%) | | | | |
| Conditioned bulk | 0.295 | 0.280 | 0.416 | 0.407 |
| density (g/cm³) | | | | |
| FFc @ 3 kPa | 3.4 (±0.52) | 5.4 (±0.39) | 6.6 (±0.64) | 13 (±0.35) |

While the traditional approach of blending with glidants such as 1% colloidal silica improved the FFc, there was no change in the bulk density or CBD values. On the other hand, coating with ALD showed significant improvement for the FFc, bulk density as well as CBD values. The flowability of the blend is critical to achieve the improved process (flowing through hopper, feeder performance, mixing efficiency, filling the die, etc.) and consistent quality tablets with acceptable critical quality attributes such as uniformity of dosage, assay, and dissolution.

Blend B and Blend D were used for tableting studies. Tablets with the target weight of 225 mg were prepared using a manual single station tablet press. To understand the impact of coating on compaction characteristics, tablets were compressed at five different compaction forces of 50, 100, 150, 200 and 250 MPa. Intact shinny tablets were manufactured. Minor surface picking was observed for some tablets for the uncoated and coated blends at lower compaction pressures (50 and 100 MPa). Such behavior was not observed for the tablets compressed at higher compaction pressures of 150 MPa.

The tabletability profile of the resultant tablets (FIG. 4, upper line=coated particle blend; lower liner=uncoated particle blend) showed the desired linear increase in tensile strength as a function of compaction pressure. Tablets with desired tensile strength (>2 MPa) were achieved with compaction pressure ≥150 MPa. Tablets formed from the blend containing the zinc oxide coated API, showed higher tensile strength compared to that containing uncoated API at all the ranges of compaction pressures investigated in this study. In summary, ALD coating of API imparted beneficial impact on the flow and tableting properties of the blend.

### Example 2

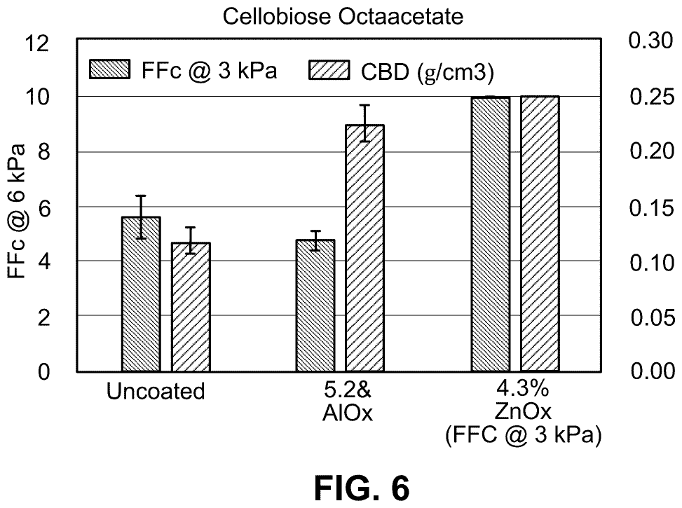

Cellobiose octaacetate was coated by ALD essentially as described above to produce an aluminum oxide coating or a zinc oxide coating. FIG. 6 presents the specific surface area (SSA), conditioned bulk density (CBD) and flow function coefficient (FFc). The FFc was measured at 3 kPa. Both the aluminum oxide coating and the zinc oxide coating improved flowability as assessed by FFc.

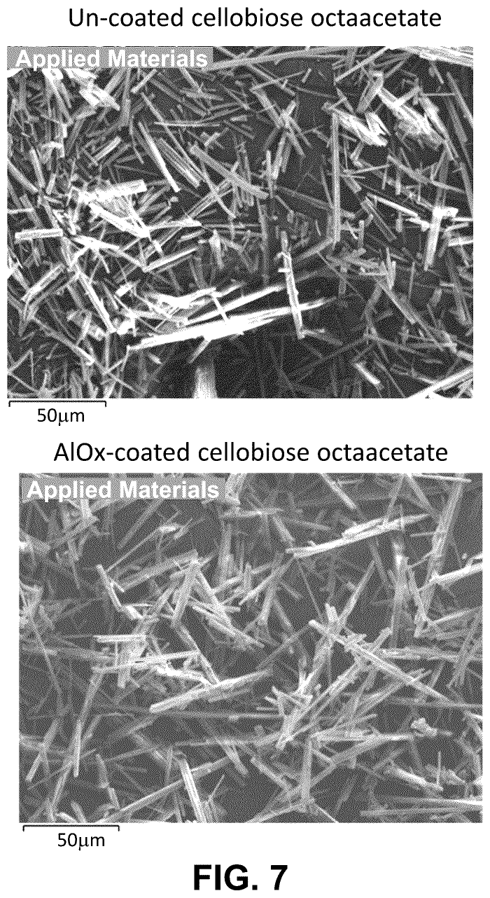

FIG. 7 presents scanning electron micrograph images of the coated and uncoated particles of Compound Alpha and Compound B characterized in FIG. 2 and coated and uncoated cellobiose octaacetate.

1. A method of preparing a pharmaceutical composition comprising inorganic oxide-coated particles comprising an organic active pharmaceutical ingredient (API) enclosed by a conformal layer of at least one inorganic oxide, the method comprising the sequential steps of:
(a) providing particles comprising an organic active pharmaceutical ingredient wherein the particles have an aspect ratio (maximum dimension/minimum dimension) greater than 5;
(b) performing atomic layer deposition to apply at least one inorganic oxide layer selected from metal oxides and metalloid oxides to the particles comprising an organic active pharmaceutical ingredient thereby preparing inorganic oxide-coated particles comprising an organic active pharmaceutical ingredient enclosed by one or more inorganic oxide layers; and
(c) processing the coated particles to prepare a pharmaceutical composition.

2. The method of claim 1, wherein the particles have an aspect ratio of between 5 and 10, 5 and 20, 5 and 50 or greater than 40.

3. The method of claim 2, wherein the particles consist of a crystalline API or an amorphous API.

4. The method of claim 1, wherein the particles have a D50 of 0.1 μm to 100 μm on a volume average basis.

5. (canceled)

6. (canceled)

7. The method of claim 1, wherein the particles are dendrites having at least one branch.

8. (canceled)

9. The method of claim 7, wherein the uncoated particles have an average of at least 2, 3, 4 or 5 branches.

10. The method of claim 1, wherein the inorganic oxide coating is 1-10 nM thick on average.

11. The method of claim 1, wherein the specific surface area of the particles is: greater than 2 m²/g, greater than 4 m²/g, greater than 6 m²/g, greater than 8 m²/g, or between 2 or 4 and 8 m²/g.

12. (canceled)

13. (canceled)

14. (canceled)

15. (canceled)

16. The method of claim 1, wherein the step of performing atomic layer deposition comprises:
(b1) loading the particles comprising the organic API into a reactor;
(b2) applying a vaporous or gaseous metal or metalloid precursor to the particles in the reactor by pulsing the vaporous or gaseous metal or metalloid precursor into the reactor at least two times;
(b3) optionally performing one or more pump-purge cycles of the reactor using inert gas;
(b4) applying a vaporous or gaseous oxidant to the particles in the reactor by pulsing the vaporous or gaseous oxidant into the reactor at least two times; and
(b5) optionally performing one or more pump-purge cycles of the reactor using inert gas.

17. (canceled)

18. (canceled)

19. (canceled)

20. The method of claim 16, wherein an average number of pulses in step (b4) of the first two cycles is greater than an average number of pulses in step (b4) of the last two cycles.

21. The method of claim 16, wherein an average number of pulses in step (b2) of the first two cycles is greater than an average number of pulses in step (b2) of the remaining cycles.

22. The method of claim 16, wherein only a portion of vapor or gaseous content is pumped out prior to step (b3) and/or step (b5).

23. (canceled)

24. (canceled)

25. (canceled)

26. (canceled)

27. (canceled)

28. The method of claim 1, wherein the inorganic oxide is selected from the group consisting of aluminum oxide, zinc oxide and titanium oxide.

29. (canceled)

30. The method of claim 1, wherein the step of further processing the coated particles to create a pharmaceutical composition comprises combining the coated particles with one or more pharmaceutically acceptable excipients.

31. The method of claim 1, wherein the step of further processing the coated particles to create a pharmaceutical composition comprises combining the coated particles with one or more pharmaceutically acceptable excipients and creating a powder, tablet or capsule or oral suspension or injectable liquid composition.

32. Coated particles having a core comprising an organic active pharmaceutical ingredient enclosed by one or more inorganic oxide layers, wherein the coated particles have an aspect ratio greater than 5.

33. The coated particles of claim 32, wherein the one or more inorganic oxide layers have a total thickness in range of 1 nm to 15 nm, 1 nm to 10 nm or 2 nm-10 nm.

34. The coated particles of claim 32, wherein the core of the coated particles comprises an organic active pharmaceutical ingredient and one or more pharmaceutically acceptable excipients.

35. The coated particles of claim 32, wherein the inorganic oxide is selected from the group consisting of aluminum oxide, silicon oxide, zinc oxide and titanium oxide and combinations thereof.

36. (canceled)

37. (canceled)

38. The coated particles of claim 32, wherein the coated particles have an aspect ratio of between 5 and 100.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)
